(19) United States
(12) Patent Application Publication
(10) Pub. No.: US 2024/0181095 A1
(43) Pub. Date: Jun. 6, 2024

(54) A DELTA-OPIOID RECEPTOR TARGETED AGENT FOR MOLECULAR IMAGING AND IMMUNOTHERAPY OF CANCER

(71) Applicant: WEST VIRGINIA UNIVERSITY BOARD OF GOVERNORS ON BEHALF OF WEST VIRGINIA UNIVERSITY, Morgantown, WV (US)

(72) Inventors: Jose Gabriel GARCIA, Gainesville, FL (US); Mark MCLAUGHLIN, Morgantown, WV (US)

(21) Appl. No.: 18/548,724

(22) PCT Filed: Mar. 1, 2022
(86) PCT No.: PCT/US2022/070893

§ 371 Date: Sep. 1, 2023

Related U.S. Application Data
(60) Provisional application No. 63/154,928, filed on Mar. 1, 2021.

(51) Int. Cl. A61K 51/10; A61K 47/68; A61K 49/00; A61K 51/04

(52) U.S. Cl. CPC A61K 51/1093; A61K 47/6803; A61K 47/6849; A61K 49/0032; A61K 49/0052; A61K 49/0058; A61K 51/0455; A61K 51/0497; A61K 51/1027

(57) ABSTRACT

This invention provides a molecular conjugate of anticancer compounds and imaging agents, generally as a cancer therapy comprising an antagonist of a cell surface opioid receptor such as a delta opioid receptor, specific to a target cell, an imaging agent, and an immune modulatory molecule, such as a T cell modulator, conjugated to the opioid receptor antagonist. The target cell can be a cell responsible for development of a disease in a subject, for example, a cancer cell. In certain embodiments, the immune modulatory molecule is an immune effector antibody. A method of treating a disease is provided comprising administering to a patient a therapeutically effective amount of the compounds or compositions of this invention to the patient. The disease may be treating cancer.

### CROSS-REFERENCE TO RELATED APPLICATION

This non-provisional patent application claims the benefit of priority to U.S. Provisional Patent Application Ser. No. 63/154,928, filed Mar. 1, 2021. The entire contents of U.S. Provisional Patent Application Ser. No. 63/154,928 is incorporated by reference into this utility non-provisional patent application as if fully rewritten herein.

### STATEMENT REGARDING FEDERALLY SPONSORED RESEARCH OR DEVELOPMENT

Not applicable.

### BACKGROUND OF THE INVENTION

### 1. Field of the Invention

The current invention pertains to a molecular conjugate of anticancer compounds and imaging agents, generally as a cancer therapy comprising an antagonist of a cell surface opioid receptor such as a delta opioid receptor, specific to a target cell, an imaging agent, and an immune effector, such as a T cell modulator, conjugated to the opioid receptor antagonist. The target cell can be a cell responsible for development of a disease in a subject, for example, a cancer cell. In certain embodiments, the immune effector is an immune effector antibody. The current invention also pertains to a method of treating a disease in a subject, the method comprising administering to the subject a pharmaceutically effective amount of the molecular conjugates of the current invention to the subject. The methods of the current invention can be used to treat cancer, such as colon cancer, breast cancer, ovarian cancer, prostate cancer, lung cancer, pancreatic cancer, or melanoma. The subject matter disclosed herein relates generally to cancer therapy and to anti-cancer compounds and imaging agents. More specifically, the subject matter disclosed herein relates to agents that target Delta Opioid Receptor (DOR) and their use in the treatment of cancer.

### 2. Background Art

Immune suppression is now recognized as one of the ten hallmarks of cancer. Most tumors are immunogenic but evade immune-mediated destruction by actively blunting the immune response.

Recently, immunotherapy agents have been approved, such as anti-CTLA4 and anti-PD1 for use in cancer and many more are being tested in clinical trials. Several of these approved agents are immune checkpoint inhibitors.

These untargeted, systemically administered immune checkpoint inhibitors are safe and effective immunotherapy agents that counteract tumor immunosuppression mechanisms. By blocking the inhibitory signal, these agents result in an activation of the immune system against the tumor. The current immune checkpoint inhibitor agents are not tumor targeted. Targeting the immunotherapy agent to specific receptors on tumor cells should concentrate the conjugate in the tumor microenvironment and enhance the immune response in the tumor while reducing the systemic dosages needed, resulting in lower out of tumor toxicity. What are needed are new, targeted agents for immunotherapies and molecular imaging of cancer. The compositions and methods disclosed herein address these and other needs.

Opioids are mainly associated with cancer as analgesics. But it is becoming increasingly clear that opioids and their receptors are an integral part of the tumor microenvironment. Opioid receptors and endogenous opioid peptides have been demonstrated in a wide variety of human tumors. Opioids may be supplied by the general circulation, produced by infiltrating leucocytes or by nerve terminals and prostatic neuroendocrine cells in the tumor microenvironment.

Considerable evidence has been accumulated, from both in vitro and in vivo studies, indicating that opioids influence tumor progression. Opioids can directly influence cancer cell invasion-associated activities such as proliferation and survival, motility and migration, cell-substrate adhesion and invasion.

### SUMMARY OF THE INVENTION

In accordance with the purposes of the disclosed materials and methods, as embodied and broadly described herein, the disclosed subject matter, in one aspect, relates to compounds, compositions and methods of making and using compounds and compositions. In specific aspects, the disclosed subject matter relates to cancer therapy and to anti-cancer compounds and imaging agents. More specifically, the subject matter disclosed herein relates to agents that target delta-opioid receptor (DOR) and their use in the treatment of cancer. The cancer may be for example, but not limited to, colon cancer, breast cancer, ovarian cancer, prostate cancer, lung cancer, pancreatic cancer, or melanoma.

Methods of screening for new agents that target DOR are also disclosed. Also disclosed are PET companion agents and their use with the disclosed compounds.

Certain embodiments of this invention provide a composition comprising at least one delta-opioid receptor targeting ligand and an immunomodulatory molecule, wherein said delta-opioid receptor targeting ligand comprising a fluorescent moiety tagged delta-opioid receptor antagonist or a rare earth compound labeled delta-opioid receptor antagonist, wherein said delta-opioid receptor targeting ligand is covalently conjugated to said immunomodulatory molecule. In another embodiment of this invention, the composition includes wherein said delta-opioid receptor antagonist is naltrindole or an analog of naltrindole. In certain other embodiments, this composition includes wherein said rare earth compound is a compound of a lanthanide series of Group MB of the Periodic Table. The rare earth compound is one selected from the group consisting of lanthanum, cerium, praseodymium, neodymium, promethium, samarium, europium, gadolinium, terbium, dysprosium, holmium, erbium, thulium, ytterbium, and lutetium.

In another embodiment, this composition, as described hereinabove, includes wherein a dodecane tetraacetic acid (DOTA) is conjugated with said rare earth compound. Certain embodiments of this invention provide wherein the composition includes wherein the delta-opioid receptor targeting ligand is naltrindole-DOTA-rare earth compound. Certain other embodiments of this invention provide wherein the composition is naltrindole-DOTA-rare earth compound-anti-PD1.

The composition of this invention includes wherein the immunomodulatory molecule is one selected from the group consisting of an antibody or a fragment of an antibody that specifically acts as an adaptive immune effector. The antibody is one selected from the group of (i) PD-1 antibody of a human, a rabbit, or a murine PD-1, (ii) an antibody that is specific for programed cell death protein 1 (anti-PD1), and (iii) one or more of CD28, CD137, OX40, and CD40 agonistic antibodies. Certain embodiments of this invention include wherein the composition comprises the immunomodulatory molecule that is an anti-PD1 checkpoint inhibitor antibody. Other embodiments of this invention include wherein the delta-opioid receptor targeting ligand is a fluorescent moiety tagged naltrindole conjugated to an anti-PD1 antibody.

In other embodiments of this invention, the composition includes wherein said immunomodulatory molecule is one that targets extracellular juxtacrine receptors. The immunomodulatory molecule may be, for example, but not limited to, scFv anti-TGIT, anti-TGFb, or a TFGb signal inhibitor.

Another embodiment of this invention provides a composition comprising at least one delta-opioid receptor targeting ligand and an immunomodulatory molecule, wherein said delta-opioid receptor targeting ligand comprising a fluorescent moiety tagged delta-opioid receptor agonist or a rare earth compound labeled delta-opioid receptor agonist, wherein said delta-opioid receptor targeting ligand is covalently conjugated to said immunomodulatory molecule.

Another embodiment of this invention provides a compound of Formula I:

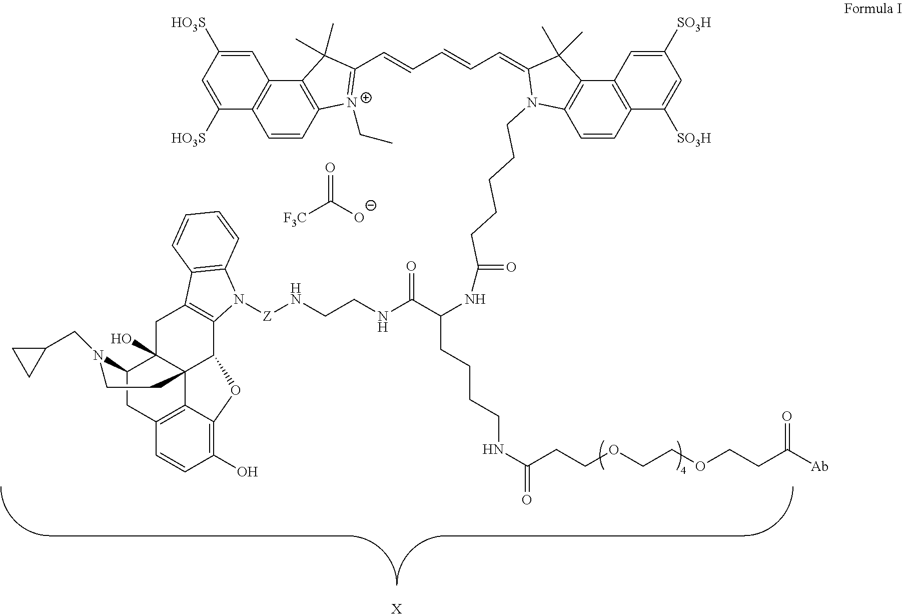

wherein
X is a delta opioid receptor targeting ligand;
Z is a linker fragment;
and Ab is an antibody; and
wherein said linker fragment Z is a single atom or multiple groups of atoms selected from the group consisting of a substituted carbon, an oxygen, a substituted or an unsubstituted Sulphur, a substituted nitrogen, a substituted phosphorous, a substituted or an unsubstituted alkyl, a substituted or an unsubstituted alkylene, a substituted alkenyl, a substituted alkynyl chain, a substituted or an unsubstituted alkyne chain, a substituted or an unsubstituted alkoxyl chain, an ether, an amine, an amide, a sulfonamide, an alkylamine, a thioether, a carboxylate, a polyethylene, a polypropylene, and a derivative or a combinations of any one or more thereof. The compound includes wherein the ratio of X to Ab is about 4 to 1. The Ab moiety is an antibody or fragment thereof that specifically acts as an adaptive immune effector, wherein said antibody is one selected from the group of (i) a PD-1 antibody of a human, a rabbit, or a murine PD-1, (ii) an antibody that is specific for programed cell death protein 1 (anti-PD1), and (iii) one or more of a CD28, a CD137, a OX40, and a CD40 agonistic antibody. The compound includes wherein said X is (X)ₙ wherein n is 4, 9 or 12. Certain embodiments of this invention provide wherein Ab is specific for programed cell death protein 1 (anti-PD1). Other embodiments provide wherein Ab is a PD-L1 antagonist, or a CD137, a OX40, or a CD40 agonist antibody. Other embodiments of this invention include wherein the compound has the Formula I wherein Ab is an antibody and X is (X)ₙ wherein n is an integer of from 1 to 50.

Another embodiment of this invention provides a compound of Formula II:

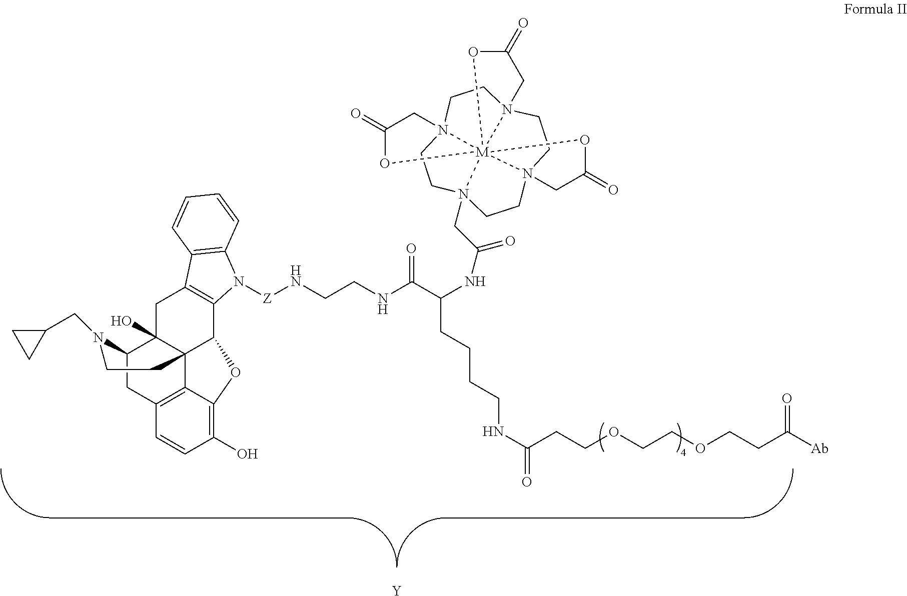

wherein
Y is a delta opioid receptor targeting ligand;
Z is a linker fragment;
M is any metal of the lanthanide series of the Periodic Table;
Ab is an antibody; and
wherein aid linker fragment Z is a single atom or multiple groups of atoms selected form the group consisting a substituted carbon, an oxygen, a substituted or an unsubstituted sulphur, a substituted nitrogen, a substituted phosphorous, a substituted or an unsubstituted alkyl, a substituted alkenyl, a substituted alkynyl chain, a substituted or an unsubstituted alkylene, a substituted or an unsubstituted alkyne chain, a substituted or an unsubstituted alkoxyl chain, an ether, an amine, an amide, a sulfonamide, an alkylamine, a thioether, a carboxylate, a polyethylene, a polypropylene, derivatives, or combinations thereof. The compound includes wherein the ratio of Y to Ab is approximately 4 to 1. The compound includes wherein said Y is (Y)ₙ wherein said n is 4, 9, or 12. Ab moiety is an antibody or fragment thereof that specifically acts as an immune effector, wherein said antibody is one selected from the group of (i) a PD-1 antibody of a human, a rabbit, or a murine PD-1, (ii) an antibody that is specific for programed cell death protein 1 (anti-PD1), and (iii) one or more of a CD28, a CD137, a OX40, and a CD40 agonistic antibody. Certain embodiments of this invention provide wherein Ab is specific for programed cell death protein 1 (anti-PD1). Other embodiments provide wherein Ab is a PD-L1 antagonist, or a CD137, a OX40, or a CD40 agonist antibody. Other embodiments of this invention include wherein the compound has the Formula II wherein Ab is an antibody and Y is (Y)ₙ wherein n is an integer of from 1 to 50.

Another embodiment of this invention provides a compound of Formula III:

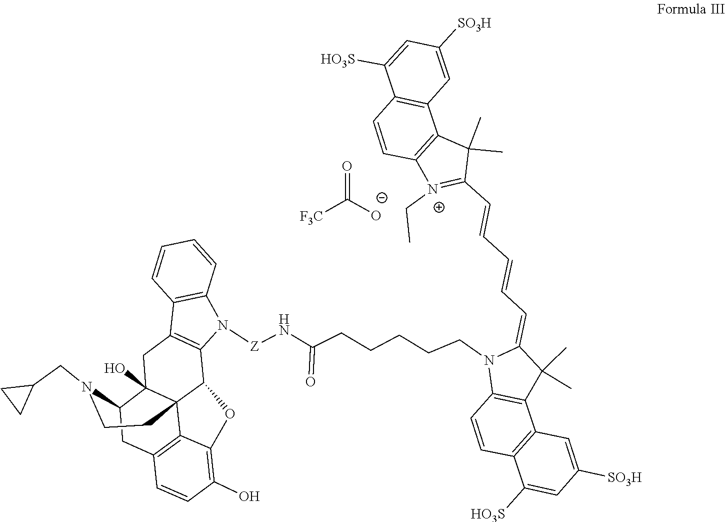

wherein
Z is a linker fragment, wherein said linker fragment Z is a single atom or multiple groups of atoms selected from the group consisting of a substituted carbon, an oxygen, a substituted or an unsubstituted sulphur, a substituted nitrogen, a substituted phosphorous, a substituted or an unsubstituted alkyl, a substituted or an unsubstituted alkylene, a substituted or an unsubstituted alkyne chain, a substituted or an unsubstituted alkoxyl chain, a substituted alkenyl, a substituted alkynyl chain, an ether, an amine, an amide, a sulfonamide, an alkylamine, a thioether, a carboxylate, a polyethylene, a polypropylene, and derivatives or combinations thereof

Another embodiment of this invention provides a compound of Formula IV:

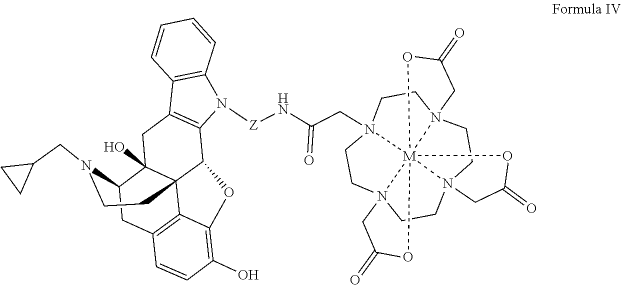

wherein
Z is a linker fragment;
M is any metal of the lanthanide series; and
wherein said linker fragment Z is a single atom or multiple groups of atoms selected from the group consisting of a substituted carbon, an oxygen, a substituted or an unsubstituted sulphur, a substituted nitrogen, a substituted phosphorous, a substituted or an unsubstituted alkyl, a substituted or an unsubstituted alkylene, a substituted or an unsubstituted alkyne chain, a substituted or an unsubstituted alkoxyl chain, a substituted alkenyl, a substituted alkynyl chain, an ether, an amine, an amide, a sulfonamide, an alkylamine, a thioether, a carboxylate, a polyethylene, a polypropylene, and derivatives or combinations thereof.

Another embodiment of this invention provides an antibody conjugated to one or more moieties of X:

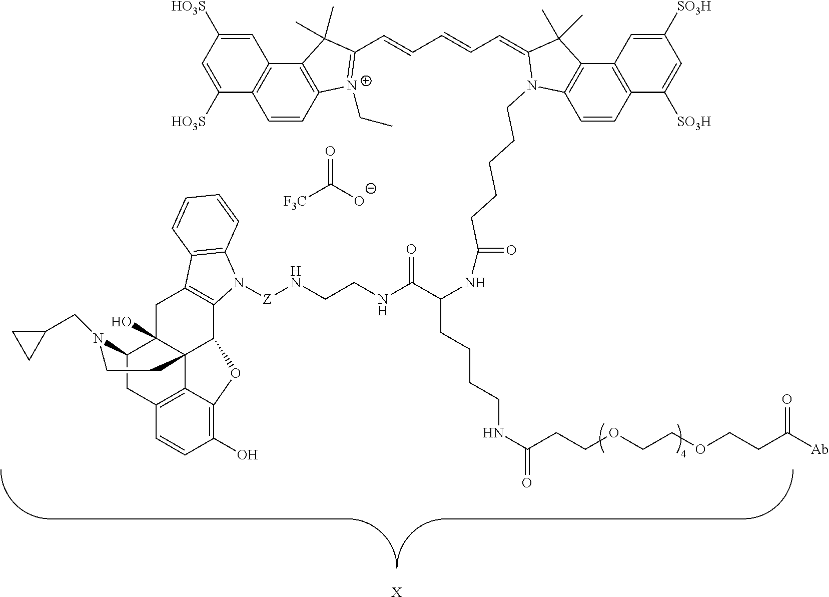

wherein
X is a delta opioid receptor targeting ligand;
Z is a linker fragment;
Ab is an antibody;
wherein said linker fragment Z is a single atom or multiple groups of atoms selected from the group consisting of a substituted carbon, an oxygen, a substituted or an unsubstituted sulphur, a substituted nitrogen, a substituted phosphorous, a substituted or an unsubstituted alkyl, a substituted or an unsubstituted alkylene, a substituted alkenyl, a substituted alkynyl chain, a substituted or an unsubstituted alkyne chain, a substituted or an unsubstituted alkoxyl chain, an ether, an amine, an amide, a sulfonamide, an alkylamine, a thioether, a carboxylate, a polyethylene, a polypropylene, and a derivative or a combinations of any one or more thereof; and X is (X)ₙ wherein n is an integer of from 1 to 50. In certain embodiments of this invention, this antibody is specific for programed cell death protein 1 (anti-PD1). This antibody is specific for a programed cell death protein 1 (PD1), a PD-L1 antagonist, or a CD137, a OX40, or a CD40 agonist antibody.

Another embodiment of this invention provides an antibody conjugated to one or more moieties of Y:

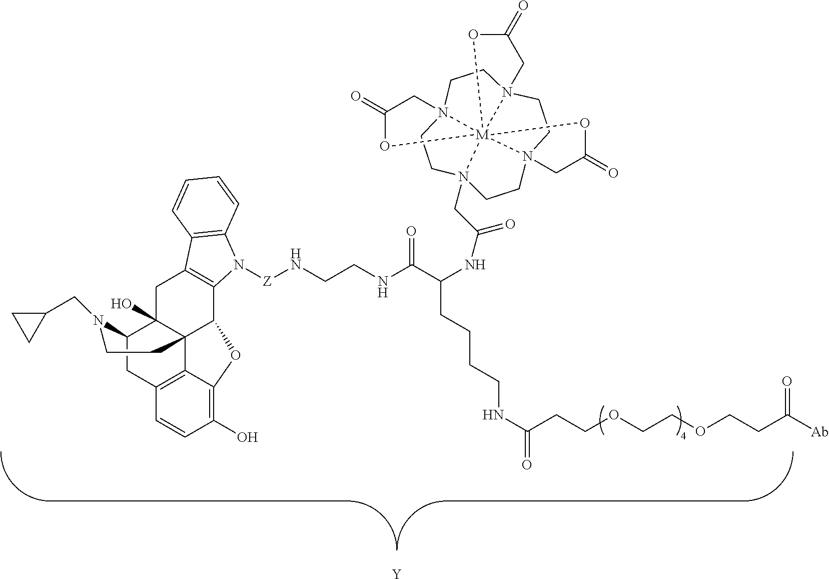

wherein
Y is a delta opioid receptor targeting ligand;
Z is a linker fragment;
M is any metal of the lanthanide series of the Periodic Table;
wherein said linker fragment Z is a single atom or multiple groups of atoms selected form the group consisting a substituted carbon, an oxygen, a substituted or an unsubstituted sulphur, a substituted nitrogen, a substituted phosphorous, a substituted or an unsubstituted alkyl, a substituted alkenyl, a substituted alkynyl chain, a substituted or an unsubstituted alkylene, a substituted or an unsubstituted alkyne chain, a substituted or an unsubstituted alkoxyl chain, an ether, an amine, an amide, a sulfonamide, an alkylamine, a thioether, a carboxylate, a polyethylene, a polypropylene, derivatives, or combinations thereof; Y is (Y)ₙ wherein n is an integer of from 1 to 50; and Ab is an antibody. In certain embodiments of this invention, this antibody is specific for programed cell death protein 1 (anti-PD1). This antibody is specific for programed cell death protein 1 (PD1), a PD-L1 antagonist, or a CD137, an OX40, or a CD40 agonist antibody.

Another embodiment of this invention provides a method of treating cancer in a patient comprising administering to a patient a therapeutically effective amount of a compound or composition of any one of the above described compounds and compositions for treating said patient. The method includes wherein the cancer is selected from the group consisting of colon cancer, lung cancer, and liver cancer. The lung cancer includes small cell lung cancer and non-small cell lung cancer.

Another embodiment of this invention provides a method of decreasing peritoneal metastasis formation in a patient comprising administering to a patient a therapeutically effective amount of a compound or composition as described herein-above for decreasing peritoneal metastasis. This method includes wherein said metastasis is distal metastasis.

Additional advantages will be set forth in part in the description that follows or may be learned by practice of the aspects described below. The advantages described below will be realized and attained by elements and combinations particularly pointed out in the appended claims. It is to be understood that both the foregoing general description and the following detailed description are exemplary and explanatory only and are not restrictive.

### DETAILED DESCRIPTION OF THE INVENTION

The materials, compounds, compositions, and methods described herein may be understood more readily by reference to the following detailed description of specific aspects of the disclosed subject matter and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

In this specification and in the claims that follow, reference will be made to several terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used herein, the singular forms “a,” “an,” and “the” include plural referents unless the context clearly dictates otherwise. Thus, for example, reference to “a composition” includes mixtures of two or more such compositions, reference to “an inhibitor” includes mixtures of two or more such inhibitors, reference to “the kinase” includes mixtures of two or more such kinase, and the like. “Optional” or “optionally” means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Unless stated otherwise, the term “about” means within 5% (e.g., within 2% or 1%) of the particular value modified by the term “about.”

By “reduce” or other forms of the word, such as “reducing” or “reduction,” is meant lowering of an event or characteristic (e.g., tumor growth, metastasis). It is understood that this is typically in relation to some standard or expected value, in other words it is relative, but that it is not always necessary for the standard or relative value to be referred to. For example, “reduces tumor growth” means decreasing the amount of tumor cells relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as tumor growth or metastasis), diminishment of extent of cancer, stabilized (i.e., not worsening) state of cancer, delaying spread (e.g., metastasis) of the cancer, delaying occurrence or recurrence of cancer, delay or slowing of cancer progression, amelioration of the cancer state, and remission (whether partial or total).

The term “patient” preferably refers to a human in need of treatment with an anti-cancer agent or treatment for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an anti-cancer agent or treatment.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5 and are present in such ratio regardless of whether additional components are contained in the mixture.

A weight percent (wt. %) of a component, unless specifically stated to the contrary, is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

### Compositions, Compounds, Antibodies, and Methods

The delta opioid receptor (DOR) has been reported to be overexpressed in some lung cancers and not in normal lung.(1-6) Expression of the DOR in lung cancer patient samples by immunohistochemical staining of a tissue microarray has been validated. In addition, the synthesis of fluorescent-labeled DOR-targeted imaging agents (DORL-Cy5 and DORL-800) based on a synthetic peptide antagonist (Dmt-Tic) have been previously reported. These agents have high DOR binding affinity in vitro, demonstrate selectivity for the DOR in vitro and in vivo, and exhibit good pharmacokinetic and biodistribution profiles in vivo. Thus, it has been decided to develop lung cancer-specific immunotherapy agents targeting the DOR by conjugating non-peptidic DORL antagonists to immunomodulatory molecules mimicking the synthetic peptide.(7) Certain embodiments of this invention provide a composition comprising a fluorescently labeled or rare earth labeled DOR targeting ligand based-on functionalized Naltrindole which is covalently conjugated to immunomodulatory molecule such as for example but not limited to Nal-FT-anti-PD1 or other immune effector or Nal-DOTA-anti-PD1 or other immune effector, where Nal represents naltrindole or an analog of naltrindole, that maintain DOR antagonist activity and high affinity binding to the delta opioid receptor. The FT refers to a fluorescent tag to allow in vitro and in vivo imaging in small animals and measurement of the Nal targeting ligand ratio to the antibody or other immune effector protein. DOTA is dodecane tetraacetic acid and is an organic compound. DOTA is a complexing agent especially with lanthanide series ions (i.e. rare earth elements) forming a DOTA-rare earth conjugate. The DOTA conjugate enables a rare earth label of the Nal-immune effector conjugate such that ICP-MS analysis can be used to measure the number of Nal-targeting ligands attached to an immune effector. Alternatively, the DOTA conjugate enables radionuclide chelation for use as PET, SPECT, or other radiographic detection and imaging in small animals or humans.

Formula A below shows the structure of naltrindole and various R-substituted analogs of naltrindole:

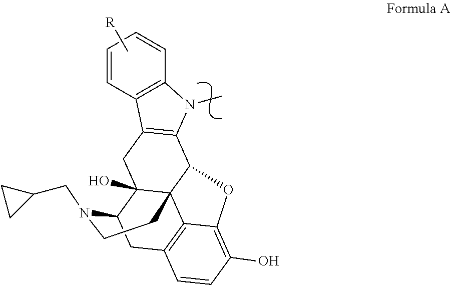

| | R |
|---|---|
| 1 | H (NTI) |
| 2 | 5′-NCS (NTII) |
| 4 | 4′-C₆H₅ |
| 5 | 4′-OC₆H₅ |
| 6 | 4′-OCH₂C₆H₅ |
| 7 | 5′-C₆H₅ |
| 8 | 5′-OC₆H₅ |
| 9 | 5′-OCH₂C₆H₅ |
| 10 | 6′-C₆H₅ |
| 11 | 6′-OC₆H₅ |
| 12 | 6′-OCH₂C₆H₅ |
| 13 | 7′-C₆H₅ |
| 14 | 7′-OC₆H₅ |
| 15 | 7′-OCH₂C₆H₅ |
| 16 | 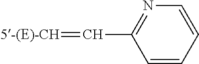 |
| 17 | 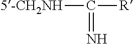 |

Regarding Formula A, compound 1 is naltrindole (“NTI”) and is represented in Formula A when R is hydrogen. Those persons of ordinary skill in the art will appreciate that Compounds 2-17 of Formula A are examples of R-substituted analogs of naltrindole. As used herein, “an analog of naltrindole” or “analogs of naltrindole” are not limited to those analog compounds represented by Formula A, and may include many additions or substitutions of elements, groups, or moieties to the chemical structure of naltrindole.

Immunoconjugates are synthesized with several targeting ligand-to-antibody ratios (TAR). These immunoconjugates are evaluated for differences in binding affinity. 344 and LKR murine lung cancer cells were engineered to constitutively express the murine DOR. Clones of the lung cancer cell lines are screened for expression of the DOR gene using qRT-PCR. Expression of DOR protein is analyzed using confocal microscopy and LTRF competitive binding assays. The binding affinity of DORL4-PD1 is evaluated in the 344/DOR cells using LTRF competitive binding assays. The binding and uptake of DORL4-PD1 in vitro is characterized using live-cell fluorescence microscopy.

In specific aspects, disclosed are “fragment” compounds required to build target compound of Formula I and II:

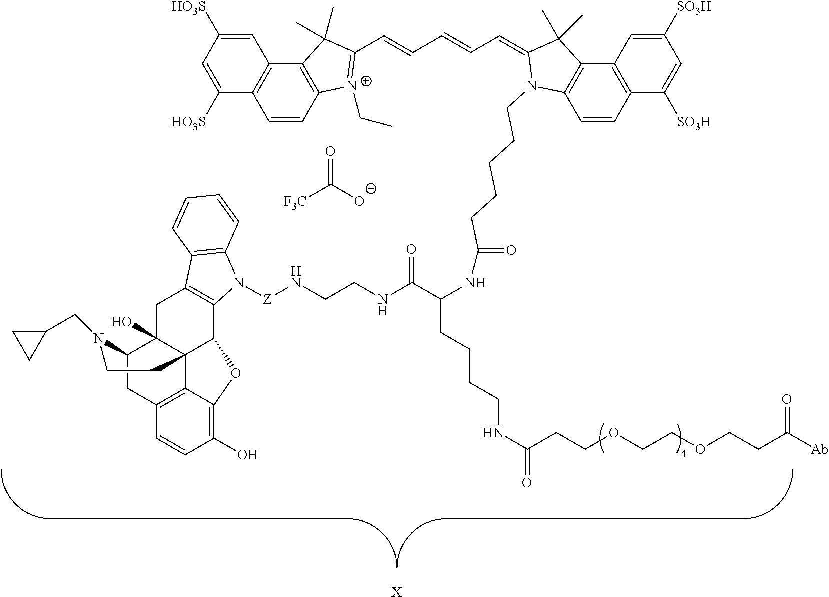

Target compound of Formula I,

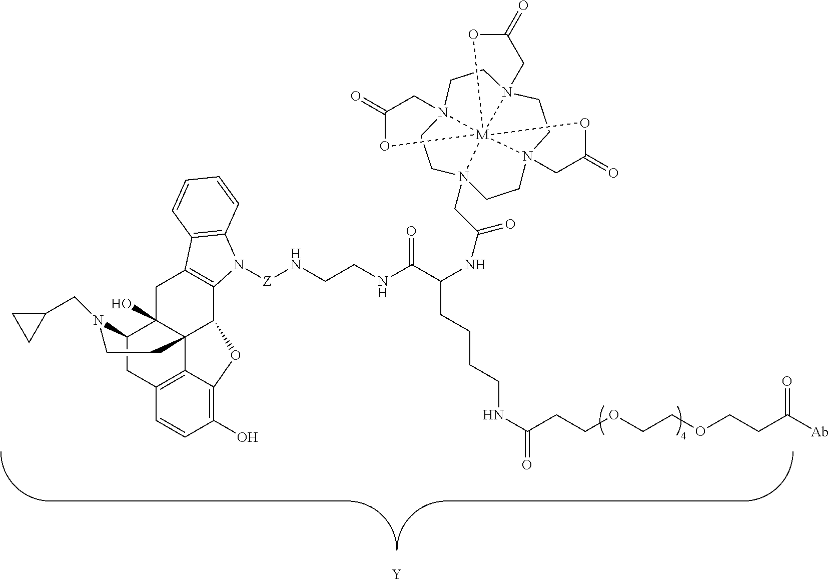

Target compound of Formula II,

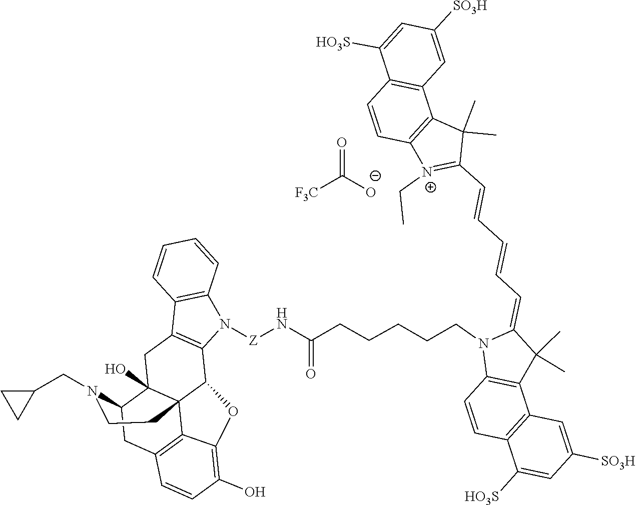

Fragment compound of Formula III, and

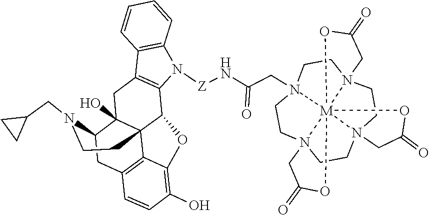

Fragment compound of Formula IV.

Certain embodiments of this invention provide a naltrindole and fluorescent tag or rare-earth metal DOTA chelate attached to an antibody via different linkers to maximize the potency of the activity of the attached antibody.

As used in the formula set forth herein, Z is a linker fragment; M is any metal of the lanthanide series; and Ab is an antibody, e.g., an antibody that can enhance an adaptive immune response such as anti-programed cell death protein 1 (anti-PD1). The target compounds described herein contain a linker that connects the DOR-targeting naltrindole to the antibody moiety via varied chain lengths.

The term “linker fragment”, as used herein, refers to one or more polyfunctional, e.g., bi-functional, or tri-functional molecules. The linker fragment “Z” can be a single atom or multiple groups of atoms, such as a substituted carbon, oxygen, substituted or unsubstituted sulphur, substituted nitrogen, substituted phosphorous, a substituted or unsubstituted alkyl, substituted or unsubstituted alkylene or substituted or unsubstituted alkyne chain or substituted or unsubstituted alkoxyl chain. Suitable linkers include but are not limited to substituted alkyl, substituted alkenyl, substituted alkynyl chains, ether, amine, amide, sulfonamide, alkylamine, thioether, carboxylates, polyethylene, polypropylene, derivatives, or combinations thereof.

The Ab moiety is an antibody or fragment thereof that specifically acts as an immune effector. PD-1 antibodies are commercially available for human, rabbit, or murine anti-PD-1. Other antibodies can be used in other embodiments, such as CD28, CD137, OX40, and CD40 agonistic antibodies.

In specific examples, disclosed herein are compounds of Formula I and II, wherein the fragment compound X and Y is approximately 4, 9 or 12. That is the ratio of DOR ligand with detectably moiety to antibody Ab is approximately 4 to 1.

In specific examples, disclosed are fluorescent delta opioid receptor (DOR)-targeted immunotherapy agents with different targeting ligands-to antibody ratios (TARs). These agents have high affinity for the DOR in vitro with higher TARs resulting in higher binding affinity. Future studies will evaluate DORL-PD1 in vivo in immunocompetent mice. These agents could be useful for molecular imaging and immunotherapy of lung cancer.

Further provided herein are methods of treating or preventing cancer in a subject, comprising administering to the subject an effective amount of a compound or composition as disclosed herein. The methods can further comprise administering a second compound or composition, such as, for example, anticancer agents or anti-inflammatory agents. Additionally, the method can further comprise administering an effective amount of ionizing radiation to the subject.

Methods of killing a tumor cell are also provided herein. The methods comprise contacting a tumor cell with an effective amount of a compound or composition as disclosed herein. The methods can further include administering a second compound or composition (e.g., an anticancer agent or an anti-inflammatory agent) or administering an effective amount of ionizing radiation to the subject.

Also provided herein are methods of radiotherapy of tumors, comprising contacting the tumor with an effective amount of a compound or composition as disclosed herein and irradiating the tumor with an effective amount of ionizing radiation.

Also disclosed are methods for treating oncological disorders in a patient. In one embodiment, an effective amount of one or more compounds or compositions disclosed herein is administered to a patient having an oncological disorder and who is in need of treatment thereof. The disclosed methods can optionally include identifying a patient who is or can be in need of treatment of an oncological disorder. The patient can be a human or other mammal, such as a primate (monkey, chimpanzee, ape, etc.), dog, cat, cow, pig, or horse, or other animals having an oncological disorder. The compounds disclosed herein are particularly suited for patients with lung cancer. However, other oncological disorders that are characterized by expression of DOR can be treated. Specific examples of such oncological disorders include, but are not limited to, cancer and/or tumors of the anus, bile duct, bladder, bone, bone marrow, bowel (including colon and rectum), breast, eye, gall bladder, kidney, mouth, larynx, esophagus, stomach, testis, cervix, head, neck, ovary, mesothelioma, neuroendocrine, penis, skin, spinal cord, thyroid, vagina, vulva, uterus, liver, muscle, pancreas, prostate, blood cells (including lymphocytes and other immune system cells), and brain. Specific cancers contemplated for treatment include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma.

Other examples of cancers that can be treated according to the methods disclosed herein are adrenocortical carcinoma, adrenocortical carcinoma, cerebellar astrocytoma, basal cell carcinoma, bile duct cancer, bladder cancer, bone cancer, brain tumor, breast cancer, Burkitt's lymphoma, carcinoid tumor, central nervous system lymphoma, cervical cancer, chronic myeloproliferative disorders, colon cancer, cutaneous T-cell lymphoma, endometrial cancer, ependymoma, esophageal cancer, gallbladder cancer, gastric (stomach) cancer, gastrointestinal carcinoid tumor, germ cell tumor, glioma, hairy cell leukemia, head and neck cancer, hepatocellular (8) cancer, hypopharyngeal cancer, hypothalamic and visual pathway glioma, intraocular melanoma, retinoblastoma, islet cell carcinoma (endocrine pancreas), laryngeal cancer, lip and oral cavity cancer, liver cancer, medulloblastoma, Merkel cell carcinoma, squamous neck cancer with occult mycosis fungoides, myelodysplastic syndromes, myelogenous leukemia, nasal cavity and paranasal sinus cancer, nasopharyngeal cancer, neuroblastoma, non-small cell lung cancer, oral cancer, oropharyngeal cancer, osteosarcoma, ovarian cancer, pancreatic cancer, paranasal sinus and nasal cavity cancer, parathyroid cancer, penile cancer, pheochromocytoma, pineoblastoma and supratentorial primitive neuroectodermal tumor, pituitary tumor, plasma cell neoplasm/multiple myeloma, pleuropulmonary blastoma, prostate cancer, rectal cancer, renal cell (kidney) cancer, retinoblastoma, rhabdomyosarcoma, salivary gland cancer, Ewing's sarcoma, soft tissue sarcoma, Sezary syndrome, skin cancer, small cell lung cancer, small intestine cancer, supratentorial primitive neuroectodermal tumors, testicular cancer, thymic carcinoma, thymoma, thyroid cancer, transitional cell cancer of the renal pelvis and ureter, trophoblastic tumor, urethral cancer, uterine cancer, vaginal cancer, vulvar cancer, Waldenstrom's macroglobulinemia, and Wilms' tumor.

In specific examples, the cancer is non-small cell or small cell lung cancer, which are known to overexpress DOR. Other cancers overexpress DOR such as liver cancer and can be targeted as described for lung cancer.(6)

Certain embodiments of this invention provide a composition comprising at least one delta-opioid receptor targeting ligand and an immunomodulatory molecule, wherein said delta-opioid receptor targeting ligand comprising a fluorescent moiety tagged delta-opioid receptor antagonist or a rare earth compound labeled delta-opioid receptor antagonist, wherein said delta-opioid receptor targeting ligand is covalently conjugated to said immunomodulatory molecule. In another embodiment of this invention, the composition includes wherein said delta-opioid receptor antagonist is naltrindole or an analog of naltrindole. In certain other embodiments, this composition includes wherein said rare earth compound is a compound of a lanthanide series of Group IIIB of the Periodic Table. The rare earth compound is one selected from the group consisting of lanthanum, cerium, praseodymium, neodymium, promethium, samarium, europium, gadolinium, terbium, dysprosium, holmium, erbium, thulium, ytterbium, and lutetium.

In another embodiment, this composition, as described hereinabove, includes wherein a dodecane tetraacetic acid (DOTA) is conjugated with said rare earth compound. Certain embodiments of this invention provide wherein the composition includes wherein the delta-opioid receptor targeting ligand is naltrindole-DOTA-rare earth compound. Certain other embodiments of this invention provide wherein the composition is naltrindole-DOTA-rare earth compound-anti-PD1.

The composition of this invention includes wherein the immunomodulatory molecule is one selected from the group consisting of an antibody or a fragment of an antibody that specifically acts as an adaptive immune effector. The antibody is one selected from the group of (i) PD-1 antibody of a human, a rabbit, or a murine PD-1, (ii) an antibody that is specific for programed cell death protein 1 (anti-PD1), and (iii) one or more of CD28, CD137, OX40, and CD40 agonistic antibodies. Certain embodiments of this invention include wherein the composition comprises the immunomodulatory molecule that is an anti-PD1 checkpoint inhibitor antibody. Other embodiments of this invention include wherein the delta-opioid receptor targeting ligand is a fluorescent moiety tagged naltrindole conjugated to an anti-PD1 antibody.

In other embodiments of this invention, the composition includes wherein said immunomodulatory molecule is one that targets extracellular juxtacrine receptors. The immunomodulatory molecule may be, for example, but not limited to, scFv anti-TGIT, anti-TGFb, or a TFGb signal inhibitor.

Another embodiment of this invention provides a composition comprising at least one delta-opioid receptor targeting ligand and an immunomodulatory molecule, wherein said delta-opioid receptor targeting ligand comprising a fluorescent moiety tagged delta-opioid receptor agonist or a rare earth compound labeled delta-opioid receptor agonist, wherein said delta-opioid receptor targeting ligand is covalently conjugated to said immunomodulatory molecule.

Another embodiment of this invention provides a compound of Formula I:

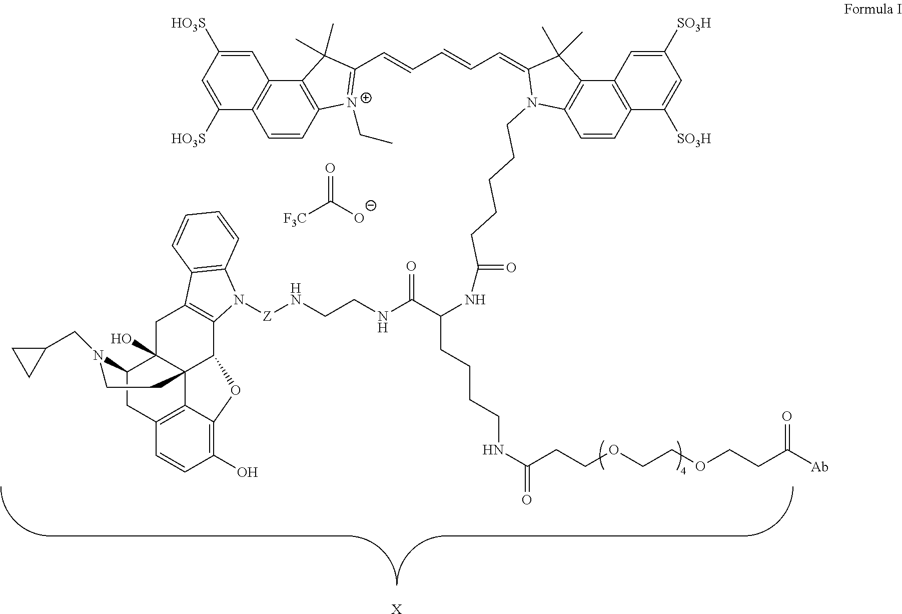

wherein
X is a delta opioid receptor targeting ligand;
Z is a linker fragment;
and Ab is an antibody; and
wherein said linker fragment Z is a single atom or multiple groups of atoms selected from the group consisting of a substituted carbon, an oxygen, a substituted or an unsubstituted sulphur, a substituted nitrogen, a substituted phosphorous, a substituted or an unsubstituted alkyl, a substituted or an unsubstituted alkylene, a substituted alkenyl, a substituted alkynyl chain, a substituted or an unsubstituted alkyne chain, a substituted or an unsubstituted alkoxyl chain, an ether, an amine, an amide, a sulfonamide, an alkylamine, a thioether, a carboxylate, a polyethylene, a polypropylene, and a derivative or a combinations of any one or more thereof. The compound includes wherein the ratio of X to Ab is about 4 to 1. The Ab moiety is an antibody or fragment thereof that specifically acts as an adaptive immune effector, wherein said antibody is one selected from the group of (i) a PD-1 antibody of a human, a rabbit, or a murine PD-1, (ii) an antibody that is specific for programed cell death protein 1 (anti-PD1), and (iii) one or more of a CD28, a CD137, a OX40, and a CD40 agonistic antibody. The compound includes wherein said X is (X)ₙ wherein n is 4, 9 or 12. Certain embodiments of this invention provide wherein Ab is specific for programed cell death protein 1 (anti-PD1). Other embodiments provide wherein Ab is a PD-L1 antagonist, or a CD137, a OX40, or a CD40 agonist antibody. Other embodiments of this invention include wherein the compound has the Formula I wherein Ab is an antibody and X is (X)ₙ wherein n is an integer of from 1 to 50.

Another embodiment of this invention provides a compound of Formula II:

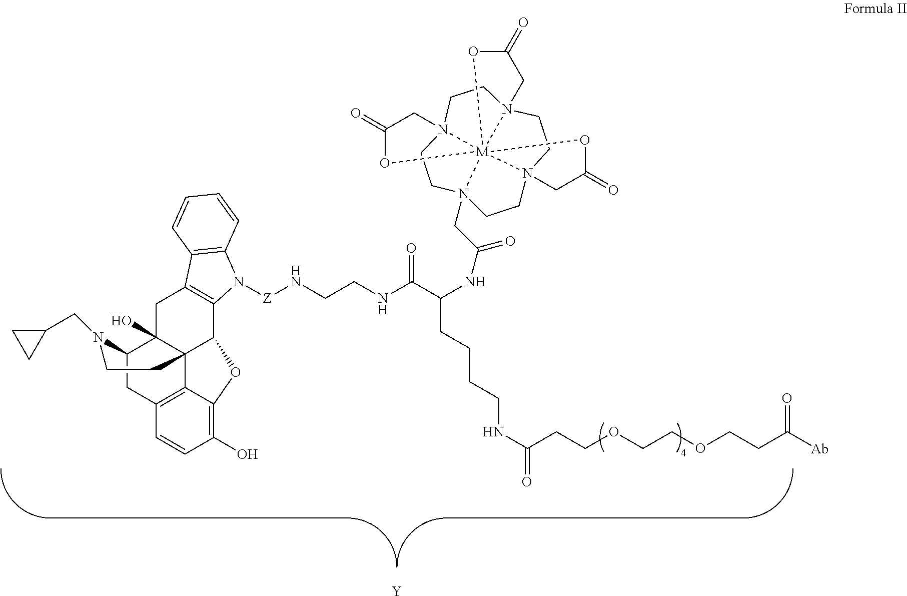

wherein
Y is a delta opioid receptor targeting ligand;
Z is a linker fragment;
M is any metal of the lanthanide series of the Periodic Table;
Ab is an antibody; and
wherein aid linker fragment Z is a single atom or multiple groups of atoms selected form the group consisting a substituted carbon, an oxygen, a substituted or an unsubstituted sulphur, a substituted nitrogen, a substituted phosphorous, a substituted or an unsubstituted alkyl, a substituted alkenyl, a substituted alkynyl chain, a substituted or an unsubstituted alkylene, a substituted or an unsubstituted alkyne chain, a substituted or an unsubstituted alkoxyl chain, an ether, an amine, an amide, a sulfonamide, an alkylamine, a thioether, a carboxylate, a polyethylene, a polypropylene, derivatives, or combinations thereof. The compound includes wherein the ratio of Y to Ab is approximately 4 to 1. The compound includes wherein said Y is (Y)ₙ wherein said n is 4, 9, or 12. Ab moiety is an antibody or fragment thereof that specifically acts as an immune effector, wherein said antibody is one selected from the group of (i) a PD-1 antibody of a human, a rabbit, or a murine PD-1, (ii) an antibody that is specific for programed cell death protein 1 (anti-PD1), and (iii) one or more of a CD28, a CD137, a OX40, and a CD40 agonistic antibody. Certain embodiments of this invention provide wherein Ab is specific for programed cell death protein 1 (anti-PD1). Other embodiments provide wherein Ab is a PD-L1 antagonist, or a CD137, an OX40, or a CD40 agonist antibody. Other embodiments of this invention include wherein the compound has the Formula II wherein Ab is an antibody and Y is (Y)ₙ wherein n is an integer of from 1 to 50.

Another embodiment of this invention provides a compound of Formula III:

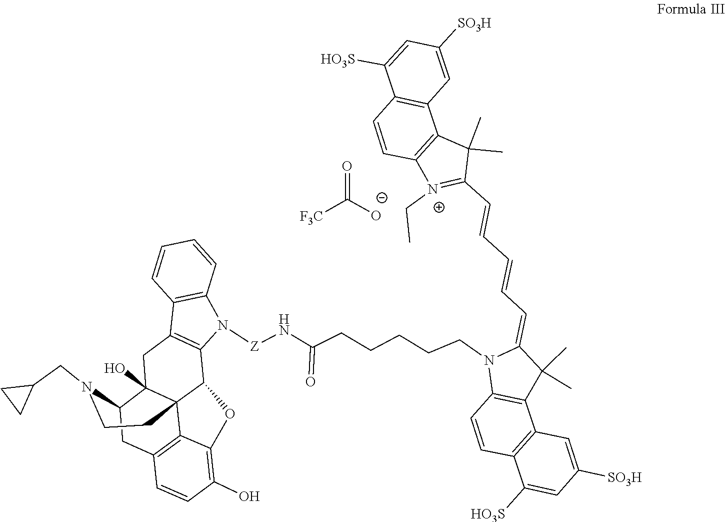

wherein
Z is a linker fragment, wherein said linker fragment Z is a single atom or multiple groups of atoms selected from the group consisting of a substituted carbon, an oxygen, a substituted or an unsubstituted sulphur, a substituted nitrogen, a substituted phosphorous, a substituted or an unsubstituted alkyl, a substituted or an unsubstituted alkylene, a substituted or an unsubstituted alkyne chain, a substituted or an unsubstituted alkoxyl chain, a substituted alkenyl, a substituted alkynyl chain, an ether, an amine, an amide, a sulfonamide, an alkylamine, a thioether, a carboxylate, a polyethylene, a polypropylene, and derivatives or combinations thereof

Another embodiment of this invention provides a compound of Formula IV:

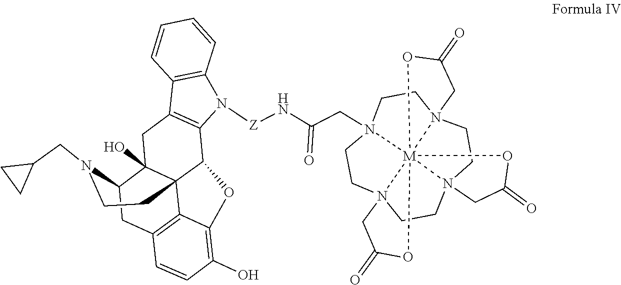

wherein
Z is a linker fragment;
M is any metal of the lanthanide series; and
wherein said linker fragment Z is a single atom or multiple groups of atoms selected from the group consisting of a substituted carbon, an oxygen, a substituted or an unsubstituted sulphur, a substituted nitrogen, a substituted phosphorous, a substituted or an unsubstituted alkyl, a substituted or an unsubstituted alkylene, a substituted or an unsubstituted alkyne chain, a substituted or an unsubstituted alkoxyl chain, a substituted alkenyl, a substituted alkynyl chain, an ether, an amine, an amide, a sulfonamide, an alkylamine, a thioether, a carboxylate, a polyethylene, a polypropylene, and derivatives or combinations thereof.

Another embodiment of this invention provides an antibody conjugated to one or more moieties of X:

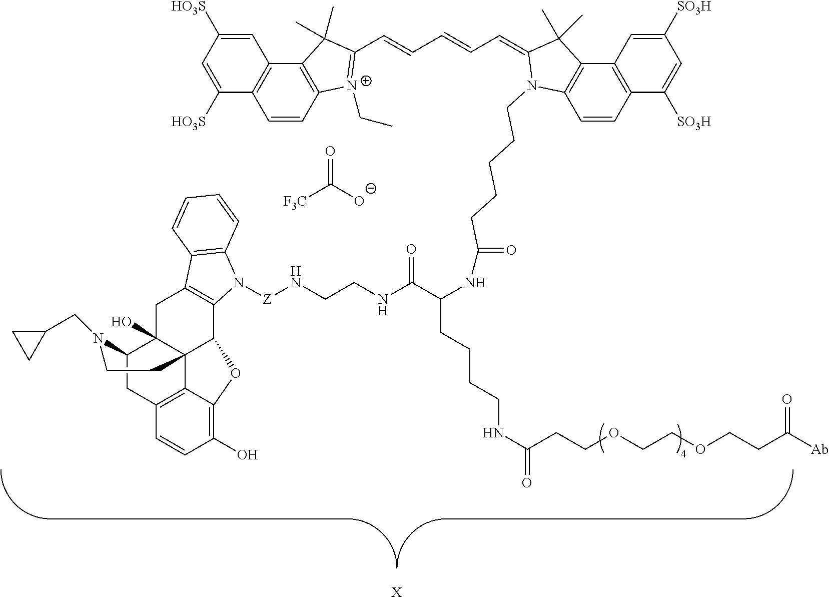

wherein
X is a delta opioid receptor targeting ligand;
Z is a linker fragment;
Ab is an antibody;
wherein said linker fragment Z is a single atom or multiple groups of atoms selected from the group consisting of a substituted carbon, an oxygen, a substituted or an unsubstituted sulphur, a substituted nitrogen, a substituted phosphorous, a substituted or an unsubstituted alkyl, a substituted or an unsubstituted alkylene, a substituted alkenyl, a substituted alkynyl chain, a substituted or an unsubstituted alkyne chain, a substituted or an unsubstituted alkoxyl chain, an ether, an amine, an amide, a sulfonamide, an alkylamine, a thioether, a carboxylate, a polyethylene, a polypropylene, and a derivative or a combinations of any one or more thereof; and X is (X)ₙ wherein n is an integer of from 1 to 50. In certain embodiments of this invention, this antibody is specific for programed cell death protein 1 (anti-PD1). This antibody is specific for a programed cell death protein 1 (PD1), a PD-L1 antagonist, or a CD137, an OX40, or a CD40 agonist antibody.

Another embodiment of this invention provides an antibody conjugated to one or more moieties of Y:

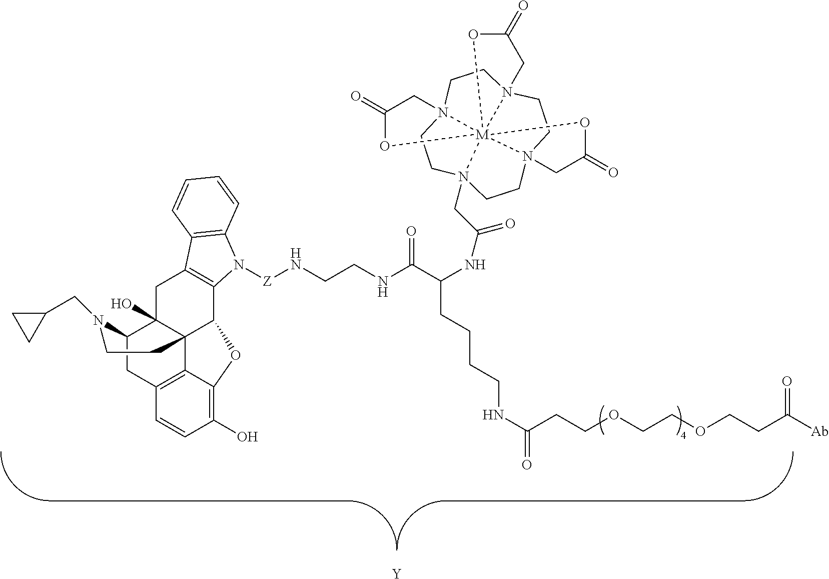

wherein
Y is a delta opioid receptor targeting ligand;
Z is a linker fragment;
M is any metal of the lanthanide series of the Periodic Table;
wherein said linker fragment Z is a single atom or multiple groups of atoms selected form the group consisting a substituted carbon, an oxygen, a substituted or an unsubstituted sulphur, a substituted nitrogen, a substituted phosphorous, a substituted or an unsubstituted alkyl, a substituted alkenyl, a substituted alkynyl chain, a substituted or an unsubstituted alkylene, a substituted or an unsubstituted alkyne chain, a substituted or an unsubstituted alkoxyl chain, an ether, an amine, an amide, a sulfonamide, an alkylamine, a thioether, a carboxylate, a polyethylene, a polypropylene, derivatives, or combinations thereof; Y is (Y)ₙ wherein n is an integer of from 1 to 50; and Ab is an antibody. In certain embodiments of this invention, this antibody is specific for programed cell death protein 1 (anti-PD1). This antibody is specific for programed cell death protein 1 (PD1), a PD-L1 antagonist, or a CD137, an OX40, or a CD40 agonist antibody.

In certain embodiments of this invention, as used herein, the terms alkyl, alkenyl, alkynyl chain, alkyne chain, alkylene, alkyamine, and alkoxyl chain may include a carbon length having one or more carbon atoms or from two to ten carbon atoms or from two to twenty carbon atoms.

Another embodiment of this invention provides a method of treating cancer in a patient comprising administering to a patient a therapeutically effective amount of a compound or composition of any one of the above-described compounds and compositions for treating said patient. The method includes wherein the cancer is selected from the group consisting of colon cancer, lung cancer, and liver cancer. The lung cancer includes small cell lung cancer and non-small cell lung cancer.

Another embodiment of this invention provides a method of decreasing peritoneal metastasis formation in a patient comprising administering to a patient a therapeutically effective amount of a compound or composition as described herein-above for decreasing peritoneal metastasis. This method includes wherein said metastasis is distal metastasis.

In some aspect, disclosed are methods for treating a tumor or tumor metastases in a subject by the administration to the subject a combination of at least one compound or composition as disclosed herein and at least one cancer immunotherapeutic agent. The disclosed compounds can be administered alone or in combination with a cancer immunotherapeutic agent. The subject can receive the therapeutic compositions prior to, during or after surgical intervention to remove all or part of a tumor. Administration may be accomplished via systemic or localized intravenous (i.v.), intraperitoneal (i.p.), subcutaneous (s.c.), intramuscular (i.m.), or direct injection into a tumor mass.

A cancer immunotherapeutic agent suitable for use in the methods disclosed herein is an immuno therapeutic agent which comprises an adaptive effector component joined to a small molecule tumor targeting ligand component. Suitable cell effector components can include cytotoxic chemicals, cytotoxic radioisotopes, and cell signaling agents such as cytokines.

An antibody with immune effector activity will have one or more non-peptidic naltrindole analogs with DOR antagonistic activity and high affinity >10 nM DOR binding affinity. The number of naltrindole targeting ligands must be at least 1 and less than the number lysine side chains on the antibody or antibody fragment. Fragments of the antibody protein, such as F(ab′)2, Fab, Fv or engineered Fv single chain antibody protein can be used. To further reduce antigenicity of the antibody or antibody sub-unit, modification of the antibody amino acid sequence may be accomplished to reduce making the protein appear more like the patients normal antibody components. For example, monoclonal murine antibody amino acid sequences can be humanized, for administration to human patients by a variety of processes for humanization of the antibody.

Specific examples of cancer immunotherapeutic agents include an antibody that specifically binds CLTA-4, such as ipilimumab (Bristol-Myers Squibb), anti-PD-1, anti-PDL1.

The disclosed compounds can also be administered with toll like receptor (TLR) agonist. TLR agonist is a ligand for a TLR selected from the group consisting of TLR1, TLR2, TLR3, TLR4, and TLR9. For example, the TLR agonist can be a ligand selected from the group consisting of Pam3CSK4, Pam3CSK4, poly I:C, Ribomunyl, and CpG ODN.

### Administration

The disclosed compounds can be administered either sequentially or simultaneously in separate or combined pharmaceutical formulations. When one or more of the disclosed compounds is used in combination with a second therapeutic agent, the dose of each compound can be either the same as or differ from that when the compound is used alone. Appropriate doses will be readily appreciated by those skilled in the art.

The term “administration” and variants thereof (e.g., “administering” a compound) in reference to a compound as described herein means introducing the compound or a prodrug of the compound into the system of the animal in need of treatment. When a compound as described herein or prodrug thereof is provided in combination with one or more other active agents (e.g., a cytotoxic agent, etc.), “administration” and its variants are each understood to include concurrent and sequential introduction of the compound or prodrug thereof and other agents.

As used herein, the term “patient” means members of the animal kingdom, including, but not limited to, human beings. As used herein, the term “having cancer” means that the patient has been diagnosed with cancer.

As used herein, the term “therapeutically effective amount” refers to that amount of any of the present compounds or compositions, or pharmaceutically acceptable salts thereof, required to bring about a desired effect in a patient. The desired effect will vary depending on the illness. For example, the desired effect may be reducing tumor size, destroying cancerous cells, and/or preventing metastasis, any one of which may be the desired therapeutic response. On its most basic level, a therapeutically effective amount is that amount needed to inhibit mitosis of a cancerous cell.

In vivo application of the disclosed compounds, and compositions containing them, can be accomplished by any suitable method and technique presently or prospectively known to those skilled in the art. For example, the disclosed compounds can be formulated in a physiologically- or pharmaceutically acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection. Administration of the disclosed compounds or compositions can be a single administration, or at continuous or distinct intervals as can be readily determined by a person skilled in the art.

The compounds disclosed herein, and compositions comprising them, can also be administered utilizing liposome technology, slow-release capsules, implantable pumps, and biodegradable containers. These delivery methods can, advantageously, provide a uniform dosage over an extended period of time. The compounds can also be administered in their salt derivative forms or crystalline forms.

The compounds disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the compounds disclosed herein can be formulated such that an effective amount of the compound is combined with a suitable carrier in order to facilitate effective administration of the compound. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically acceptable carriers and diluents which are known to those skilled in the art. Examples of carriers or diluents for use with the compounds include ethanol, dimethyl sulfoxide, glycerol, alumina, starch, saline, and equivalent carriers and diluents. To provide for the administration of such dosages for the desired therapeutic treatment, compositions disclosed herein can advantageously comprise between about 0.1% and 99%, and especially, 1 and 15% by weight of the total of one or more of the subject compounds based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use. Extemporaneous injection solutions and suspensions can be prepared from sterile powder, granules, tablets, etc. It should be understood that in addition to the ingredients particularly mentioned above, the compositions disclosed herein can include other agents conventional in the art having regard to the type of formulation in question.

Compounds disclosed herein, and compositions comprising them, can be delivered to a cell either through direct contact with the cell or via a carrier means. Carrier means for delivering compounds and compositions to cells are known in the art and include, for example, encapsulating the composition in a liposome moiety. Another means for delivery of compounds and compositions disclosed herein to a cell comprises attaching the compounds to a protein or nucleic acid that is targeted for delivery to the target cell. U.S. Pat. No. 6,960,648 and U.S. Application Publication Nos. 2003/0032594 and 2002/0120100 disclose amino acid sequences that can be coupled to another composition and that allows the composition to be translocated across biological membranes. U.S. Application Publication No. 2002/0035243 also describes compositions for transporting biological moieties across cell membranes for intracellular delivery. Compounds can also be incorporated into polymers, examples of which include poly (D-L lactide-co-glycolide) polymer for intracranial tumors; poly[bis(p-carboxyphenoxy) propane:sebacic acid] in a 20:80 molar ratio (as used in GLIADEL); chondroitin; chitin; and chitosan.

For the treatment of oncological disorders, the compounds disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the compounds disclosed herein. For example, the compounds disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and RERCEPTIN (Genentech, Inc.), respectively.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease. For example, the compounds can be used with mutant herpes simplex virus in the treatment of non-small cell lung cancer (Toyoizumi, et al., “Combined therapy with chemotherapeutic agents and herpes simplex virus type IICP34.5 mutant (HSV-1716) in human non-small cell lung cancer,” Human Gene Therapy, 1999, 10(18):17).

Therapeutic application of compounds and/or compositions containing them can be accomplished by any suitable therapeutic method and technique presently or prospectively known to those skilled in the art. Further, compounds and compositions disclosed herein have use as starting materials or intermediates for the preparation of other useful compounds and compositions.

Compounds and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Compounds and compositions disclosed herein can be systemically administered, such as intravenously or orally, optionally in combination with a pharmaceutically acceptable carrier such as an inert diluent, or an assimilable edible carrier for oral delivery. They can be enclosed in hard- or soft-shell gelatin capsules, can be compressed into tablets, or can be incorporated directly with the food of the patient's diet. For oral therapeutic administration, the active compound can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added.

When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices. Compounds and compositions disclosed herein, including pharmaceutically acceptable salts, hydrates, or analogs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze-drying techniques, which yield a powder of the active ingredient plus any additional desired ingredient present in the previously sterile-filtered solutions.

For topical administration, compounds and agents disclosed herein can be applied in as a liquid or solid. However, it will generally be desirable to administer them topically to the skin as compositions, in combination with a dermatologically acceptable carrier, which can be a solid or a liquid. Compounds and agents and compositions disclosed herein can be applied topically to a subject's skin to reduce the size (and can include complete removal) of malignant or benign growths, or to treat an infection site. Compounds and agents disclosed herein can be applied directly to the growth or infection site. Preferably, the compounds and agents are applied to the growth or infection site in a formulation such as an ointment, cream, lotion, solution, tincture, or the like. Drug delivery systems for delivery of pharmacological substances to dermal lesions can also be used, such as that described in U.S. Pat. No. 5,167,649.

Useful solid carriers include finely divided solids such as talc, clay, microcrystalline cellulose, silica, alumina, and the like. Useful liquid carriers include water, alcohols or glycols or water-alcohol/glycol blends, in which the compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver a compound to the skin are disclosed in U.S. Pat. Nos. 4,608,392; 4,992,478; 4,559,157; and 4,820,508.

Useful dosages of the compounds and agents and pharmaceutical compositions disclosed herein can be determined by comparing their in vitro activity, and in vivo activity in animal models. Methods for the extrapolation of effective dosages in mice, and other animals, to humans are known to the art; for example, see U.S. Pat. No. 4,938,949.

Also disclosed are pharmaceutical compositions that comprise a compound disclosed herein in combination with a pharmaceutically acceptable carrier. Pharmaceutical compositions adapted for oral, topical or parenteral administration, comprising an amount of a compound constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

For the treatment of oncological disorders, compounds and agents and compositions disclosed herein can be administered to a patient in need of treatment prior to, subsequent to, or in combination with other antitumor or anticancer agents or substances (e.g., chemotherapeutic agents, immunotherapeutic agents, radiotherapeutic agents, cytotoxic agents, etc.) and/or with radiation therapy and/or with surgical treatment to remove a tumor. For example, compounds and agents and compositions disclosed herein can be used in methods of treating cancer wherein the patient is to be treated or is or has been treated with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation; East Hanover, NJ) and HERCEPTIN (Genentech, Inc.; South San Francisco, CA), respectively. These other substances or radiation treatments can be given at the same as or at different times from the compounds disclosed herein. Examples of other suitable chemotherapeutic agents include, but are not limited to, altretamine, bleomycin, bortezomib (VELCADE), busulphan, calcium folinate, capecitabine, carboplatin, carmustine, chlorambucil, cisplatin, cladribine, crisantaspase, cyclophosphamide, cytarabine, dacarbazine, dactinomycin, daunorubicin, docetaxel, doxorubicin, epirubicin, etoposide, fludarabine, fluorouracil, gefitinib (IRESSA), gemcitabine, hydroxyurea, idarubicin, ifosfamide, imatinib (GLEEVEC), irinotecan, liposomal doxorubicin, lomustine, melphalan, mercaptopurine, methotrexate, mitomycin, mitoxantrone, oxaliplatin, paclitaxel, pentostatin, procarbazine, raltitrexed, streptozocin, tegafur-uracil, temozolomide, thiotepa, tioguanine/thioguanine, topotecan, treosulfan, vinblastine, vincristine, vindesine, vinorelbine. In an exemplified embodiment, the chemotherapeutic agent is melphalan.

Examples of suitable immunotherapeutic agents include, but are not limited to, alemtuzumab, cetuximab (ERBITUX), gemtuzumab, iodine 131 tositumomab, rituximab, trastuzamab (HERCEPTIN). Cytotoxic agents include, for example, radioactive isotopes (e.g., 1131, 1125, Y90, P32, etc.), and toxins of bacterial, fungal, plant, or animal origin (e.g., ricin, botulinum toxin, anthrax toxin, aflatoxin, jellyfish venoms (e.g., box jellyfish, etc.) Also disclosed are methods for treating an oncological disorder comprising administering an effective amount of a compound and/or agent disclosed herein prior to, subsequent to, and/or in combination with administration of a chemotherapeutic agent, an immunotherapeutic agent, a radiotherapeutic agent, or radiotherapy.

Kits

Kits for practicing the methods described herein are further provided. By “kit” is intended any manufacture (e.g., a package or a container) comprising at least one reagent, e.g., anyone of the compounds described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

To provide for the administration of such dosages for the desired therapeutic treatment, in some embodiments, pharmaceutical compositions disclosed herein can comprise between about 0.1% and 45%, and especially, 1 and 15%, by weight of the total of one or more of the compounds based on the weight of the total composition including carrier or diluents.

Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

The methods and compositions of the appended claims are not limited in scope by the specific methods and compositions described herein, which are intended as illustrations of a few aspects of the claims and any methods and compositions that are functionally equivalent are within the scope of this disclosure. Various modifications of the methods and compositions in addition to those shown and described herein are intended to fall within the scope of the appended claims. Further, while only certain representative methods, compositions, and aspects of these methods and compositions are specifically described, other methods and compositions and combinations of various features of the methods and compositions are intended to fall within the scope of the appended claims, even if not specifically recited. Thus, a combination of steps, elements, components, or constituents can be explicitly mentioned herein; however, all other combinations of steps, elements, components, and constituents are included, even though not explicitly stated.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Conjugate Synthesis:

PD-1 antibody (Ab) can be coupled to the rest of the molecule following literature methods as shown in Schemes 1 and 2.

Formula II, wherein m is a linker fragment; M is any metal of the lanthanide series, and Ab is an antibody, e.g., an antibody specific for programed cell death protein 1 (PD1). The target compounds described herein contain a linker that connects the DOR naltrindole to the antibody moiety via varied chain lengths.

The term “linker fragment”, as used herein, refers to one or more polyfunctional, e.g., bi-functional, or tri-functional molecules. The linker fragment “Z” can be a single atom or multiple groups of atoms, such as a substituted carbon, oxygen, substituted or unsubstituted sulphur, substituted nitrogen, substituted phosphorous, a substituted or unsubstituted alkyl, substituted or unsubstituted alkylene or substituted or unsubstituted alkyne chain or substituted or unsubstituted alkoxyl chain. Suitable linkers include but are not limited to substituted alkyl, substituted alkenyl, substituted alkynyl chains, ether, amine, amide, sulfonamide, alkylamine, thioether, carboxylates, polyethylene, polypropylene, derivatives, or combinations thereof.

The Ab moiety is an antibody or fragment thereof that specifically acts as an immune effector. PD-1 antibodies are commercially available for human, rabbit, or murine PD-1. Other antibodies can be used in other embodiments, such as CD28, CD137, OX40, and CD40 agonistic antibodies.

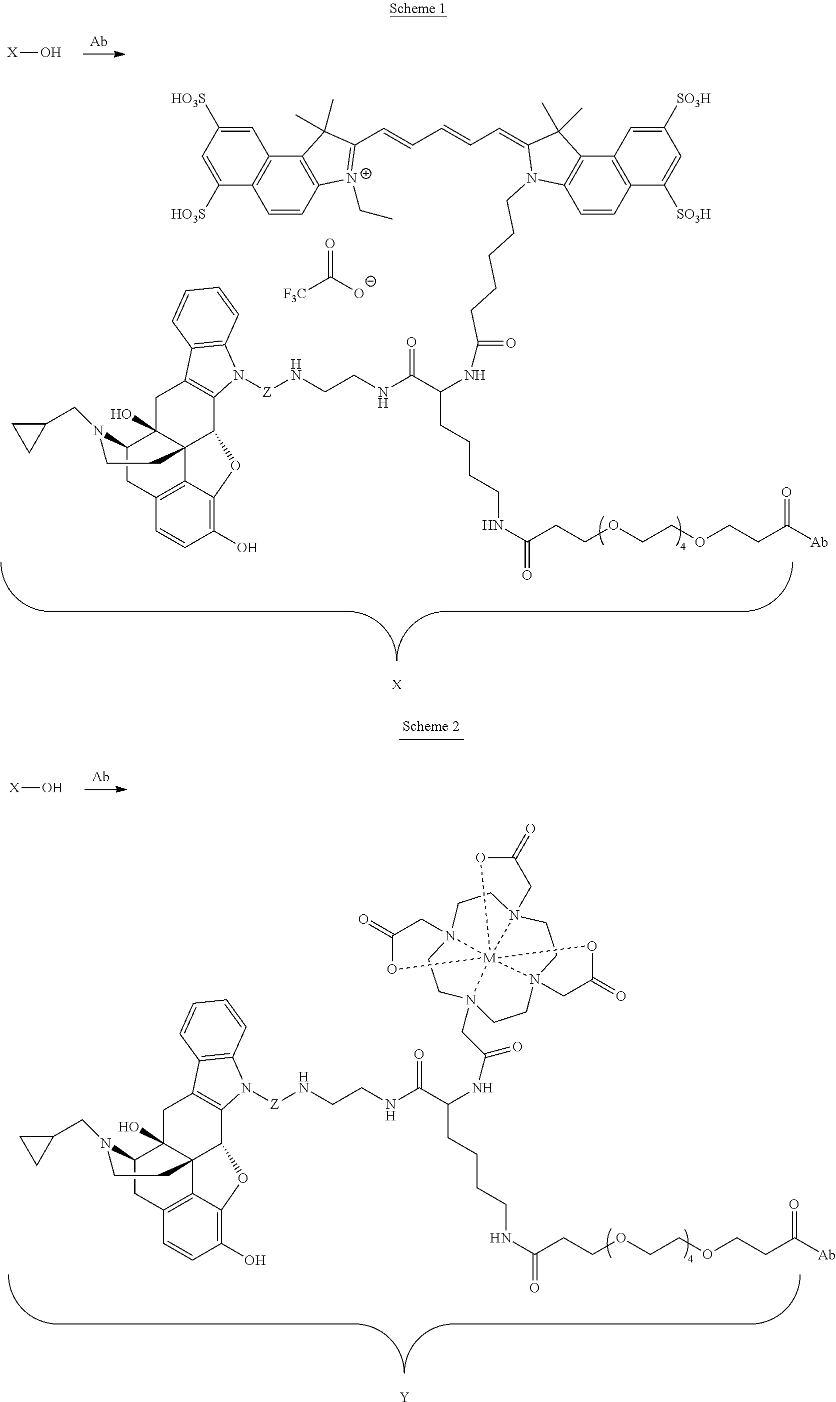

### Literature Cited

1. Cohen A S, Patek R, Enkemann S A, Johnson J O, Chen T, Toloza E, Vagner J, Morse D L. Delta-Opioid Receptor (deltaOR) Targeted Near-Infrared Fluorescent Agent for Imaging of Lung Cancer: Synthesis and Evaluation In Vitro and In Vivo. Bioconjugate chemistry. 2016;27(2):427-38. doi: 10.1021/acs.bioconjchem.5b00516. PubMed PMID: 26488422; PubMed Central PMCID: PMC5524213.
2. Huynh A S, Estrella V, Stark V E, Cohen A S, Chen T, Casagni T J, Josan J S, Lloyd M C, Johnson J, Kim J, Hruby V J, Vagner J, Morse D L. Tumor Targeting and Pharmacokinetics of a Near-Infrared Fluorescent-Labeled delta-Opioid Receptor Antagonist Agent, Dmt-Tic-Cy5. Molecular pharmaceutics. 2016;13(2):534-44. doi: 10.1021/acs.molpharmaceut.5b00760. PubMed PMID: 26713599; PubMed Central PMCID: PMC4936951.
3. Josan J S, Morse D L, Xu L, Trissal M, Baggett B, Davis P, Vagner J, Gillies R J, Hruby V J. Solid-phase synthetic strategy and bioevaluation of a labeled delta-opioid receptor ligand Dmt-Tic-Lys for in vivo imaging. Organic letters. 2009;11(12):2479-82. Epub 2009/05/19. doi: 10.1021/01900200k. PubMed PMID: 19445485; PubMed Central PMCID: PMC2756606.
4. Madar I, Bencherif B, Lever J, Heitmiller R F, Yang S C, Brock M, Brahmer J, Ravert H, Dannals R, Frost J J. Imaging delta- and mu-opioid receptors by PET in lung carcinoma patients. Journal of nuclear medicine: official publication, Society of Nuclear Medicine. 2007;48(2):207-13. Epub 2007/02/03. PubMed PMID: 17268016.
5. Schreiber G, Campa M J, Prabhakar S, O'Briant K, Bepler G, Patz E F, Jr. Molecular characterization of the human delta opioid receptor in lung cancer. Anticancer research. 1998;18(3A):1787-92. Epub 1998/07/23. PubMed PMID: 9673405.
6. Tang B, Li Y, Yuan S, Tomlinson S, He S. Upregulation of the delta opioid receptor in liver cancer promotes liver cancer progression both in vitro and in vivo. International journal of oncology. 2013;43(4):1281-90. Epub 2013/08/02. doi: 10.3892/ijo.2013.2046. PubMed PMID: 23903826.
7. McLaughlin M L, Morse D L. A delta-opioid receptor targeted agent for molecular imaging and immunotherapy of cancer. PCT Int Appl. 2017;WO 2017192798 A1 20171109.
8. Schmerzler A J, Martin L, Oliver B, London L A. Safety in the rehabilitation setting: a nursing perspective. Physical medicine and rehabilitation clinics of North America. 2012;23(2):259-70. doi: 10.1016/j.pmr.2012.02.004. PubMed PM1D: 22537692.

It will be appreciated by those persons skilled in the art that changes could be made to embodiments of the present invention described herein without departing from the broad inventive concept thereof. It is understood, therefore, that this invention is not limited by any particular embodiments disclosed, but is intended to cover the modifications that are within the spirit and scope of the invention, as defined by the appended claims.

1. A composition comprising at least one delta-opioid receptor targeting ligand and an immunomodulatory molecule, wherein said delta-opioid receptor targeting ligand is a fluorescent moiety tagged delta-opioid receptor antagonist or a rare earth compound labeled delta-opioid receptor antagonist, and wherein said delta-opioid receptor targeting ligand is covalently conjugated to said immunomodulatory molecule.

2. The composition of claim 1 wherein said delta-opioid receptor antagonist is naltrindole or an analog of naltrindole.

3. The composition of claim 1 wherein said rare earth compound is a compound of a lanthanide series of Group IIIB of the Periodic Table.

4. The composition of claim 3 wherein said rare earth compound is selected from the group consisting of lanthanum, cerium, praseodymium, neodymium, promethium, samarium, europium, gadolinium, terbium, dysprosium, holmium, erbium, thulium, ytterbium, and lutetium.

5. The composition of claim 1 wherein a dodecane tetraacetic acid (DOTA) is conjugated with said rare earth compound.

6. The composition of claim 5 wherein said delta-opioid receptor targeting ligand is naltrindole-DOTA-rare earth compound.

7. The composition of claim 6 that is naltrindole-DOTA-rare earth compound-anti-PD1.

8. The composition of claim 1 wherein said immunomodulatory molecule is selected from the group consisting of an antibody or a fragment of an antibody that specifically acts as an adaptive immune effector.

9. The composition of claim 8 wherein said antibody is selected from the group of (i) PD-1 antibody of a human, a rabbit, or a murine PD-1, (ii) an antibody that is specific for programed cell death protein 1 (anti-PD1), and (iii) one or more of a CD28, a CD137, an OX40, and a CD40 agonistic antibodies.

10. The composition of claim 8 wherein said immunomodulatory molecule is an anti-PD1 checkpoint inhibitor antibody.

11. The composition of claim 2 wherein said delta-opioid receptor targeting ligand is a fluorescent moiety tagged naltrindole conjugated to an anti-PD1 antibody.

12. The composition of claim 1 wherein said immunomodulatory molecule targets extracellular juxtacrine receptors.

13. The composition of claim 2 wherein said immunomodulatory molecule is scFv anti-TGIT, anti-TGFb, or a TFGb signal inhibitor.

14. (canceled)

15. A compound of Formula I: 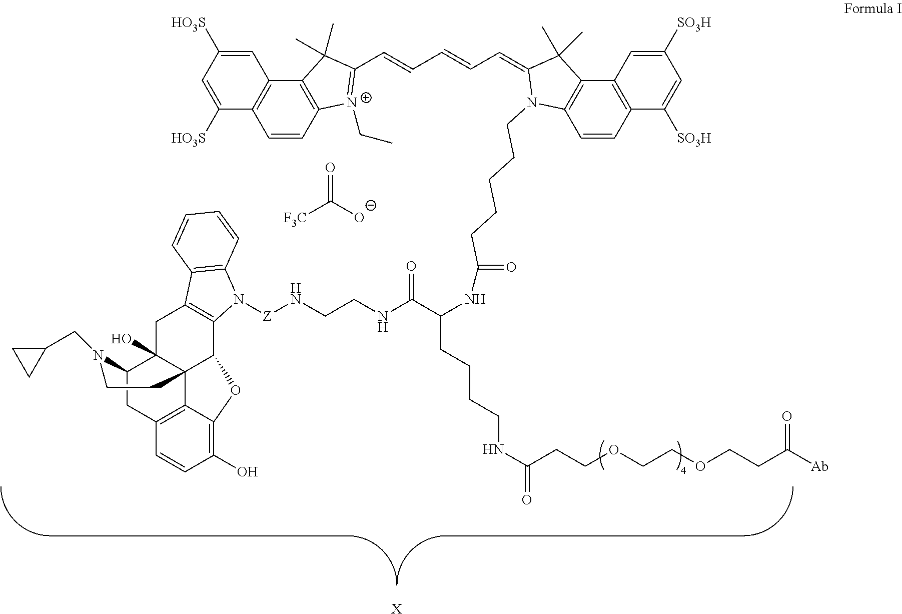
wherein
X is a delta opioid receptor targeting ligand;
Z is a linker fragment;
and Ab is an antibody; and
wherein said linker fragment Z is a single atom or is selected from the group consisting of a substituted carbon, an oxygen, a substituted or an unsubstituted sulphur, a substituted nitrogen, a substituted phosphorous, a substituted or an unsubstituted alkyl, a substituted or an unsubstituted alkylene, a substituted alkenyl, a substituted alkynyl chain, a substituted or an unsubstituted alkyne chain, a substituted or an unsubstituted alkoxyl chain, an ether, an amine, an amide, a sulfonamide, an alkylamine, a thioether, a carboxylate, a polyethylene, a polypropylene, and a derivative or a combination thereof.

16. The compound of claim 15 wherein the ratio of X to Ab is about 4 to 1.

17. The compound of claim 15 wherein the Ab moiety is an antibody or fragment thereof that specifically acts as an adaptive immune effector, wherein said antibody is one selected from the group of (i) a PD-1 antibody of a human, a rabbit, or a murine PD-1, (ii) an antibody that is specific for programed cell death protein 1 (anti-PD1), and (iii) one or more of a CD28, a CD137, an OX40, and a CD40 agonistic antibody.

18. The compound of claim 15 wherein said X is (X),, wherein n is 4, 9 or 12.

19. A compound of Formula II: 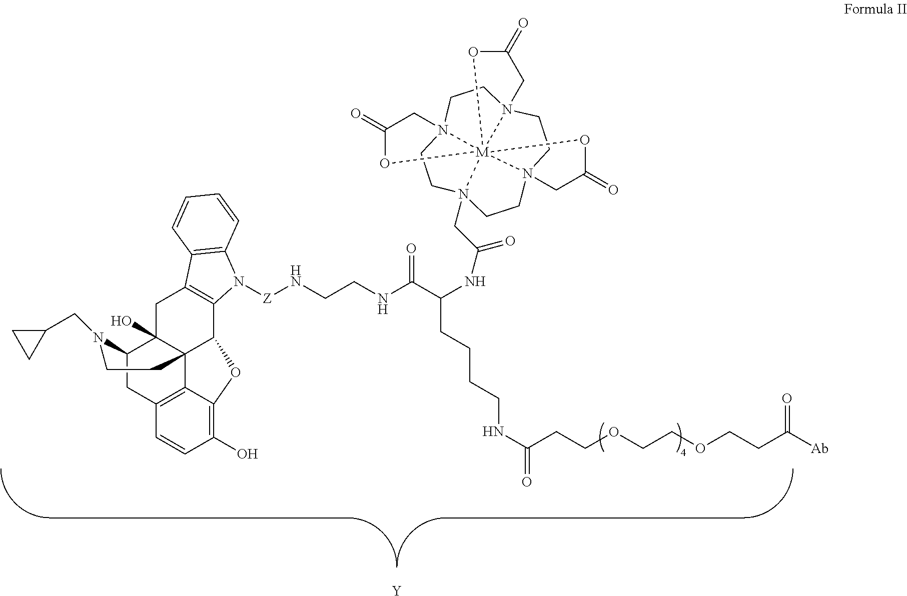
wherein
Y is a delta opioid receptor targeting ligand;
Z is a linker fragment;
M is any metal of the lanthanide series of the Periodic Table;
Ab is an antibody; and
wherein aid linker fragment Z is a single atom or is selected form the group consisting a substituted carbon, an oxygen, a substituted or an unsubstituted sulphur, a substituted nitrogen, a substituted phosphorous, a substituted or an unsubstituted alkyl, a substituted alkenyl, a substituted alkynyl chain, a substituted or an unsubstituted alkylene, a substituted or an unsubstituted alkyne chain, a substituted or an unsubstituted alkoxyl chain, an ether, an amine, an amide, a sulfonamide, an alkylamine, a thioether, a carboxylate, a polyethylene, a polypropylene, and a derivative or a combination thereof.

20. The compound of claim 19 wherein the ratio of Y to Ab is approximately 4 to 1.

21. The compound of claim 19 wherein said Y is (Y)ₙ wherein said n is 4, 9, or 12.

22. The compound of claim 19 wherein the Ab moiety is an antibody or fragment thereof that specifically acts as an immune effector, wherein said antibody is one selected from the group of (i) a PD-1 antibody of a human, a rabbit, or a murine PD-1, (ii) an antibody that is specific for programed cell death protein 1 (anti-PD1), and (iii) one or more of a CD28, a CD137, an OX40, and a CD40 agonistic antibody.

23. A compound of Formula III: 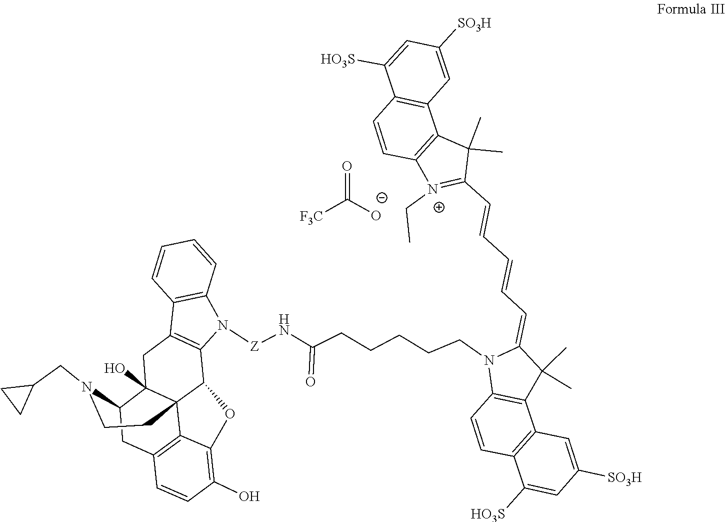
wherein
Z is a linker fragment, wherein said linker fragment Z is a single atom or is selected from the group consisting of a substituted carbon, an oxygen, a substituted or an unsubstituted sulphur, a substituted nitrogen, a substituted phosphorous, a substituted or an unsubstituted alkyl, a substituted or an unsubstituted alkylene, a substituted or an unsubstituted alkyne chain, a substituted or an unsubstituted alkoxyl chain, a substituted alkenyl, a substituted alkynyl chain, an ether, an amine, an amide, a sulfonamide, an alkylamine, a thioether, a carboxylate, a polyethylene, a polypropylene, and a derivative or a combination thereof.

24. A compound of Formula IV: 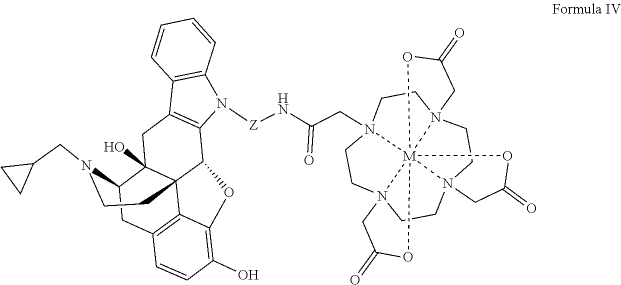
wherein
Z is a linker fragment;
M is any metal of the lanthanide series; and
wherein said linker fragment Z is a single atom or is selected from the group consisting of a substituted carbon, an oxygen, a substituted or an unsubstituted sulphur, a substituted nitrogen, a substituted phosphorous, a substituted or an unsubstituted alkyl, a substituted or an unsubstituted alkylene, a substituted or an unsubstituted alkyne chain, a substituted or an unsubstituted alkoxyl chain, a substituted alkenyl, a substituted alkynyl chain, an ether, an amine, an amide, a sulfonamide, an alkylamine, a thioether, a carboxylate, a polyethylene, a polypropylene, and a derivative or a combination thereof.

25. The compound of claim 15 wherein Ab is specific for programed cell death protein 1 (anti-PD1).

26. The compound of claim 19 wherein Ab is specific for programmed cell death protein 1 (anti-PD1).

27. The compound of claim 15 wherein Ab is a PD-L1 antagonist, or a CD137, an OX40, or a CD40 agonist antibody.

28. The compound of claim 19 wherein Ab is a PD-L1 antagonist, or a CD137, an OX40, or a CD40 agonist antibody.

29. The compound of claim 15 wherein the compound has the Formula I wherein Ab is an antibody and X is (X)ₙ wherein n is an integer of from 1 to 50.

30. The compound of claim 19 wherein the compound has the Formula II wherein Ab is an antibody and Y is (Y)ₙ wherein n is an integer of from 1 to 50.

31. An antibody conjugated to one or more moieties of X: 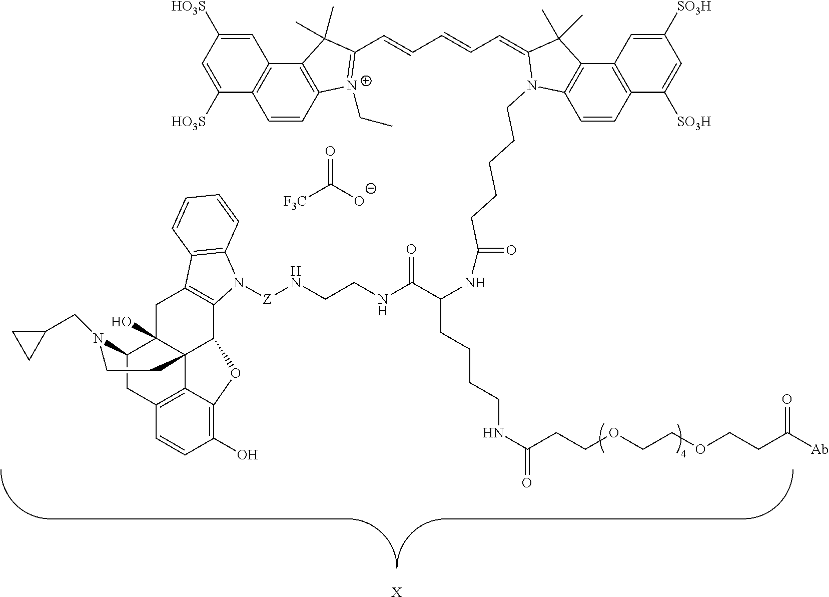
wherein
X is a delta opioid receptor targeting ligand;
Z is a linker fragment;
Ab is an antibody;
wherein said linker fragment Z is a single atom or is selected from the group consisting of a substituted carbon, an oxygen, a substituted or an unsubstituted sulphur, a substituted nitrogen, a substituted phosphorous, a substituted or an unsubstituted alkyl, a substituted or an unsubstituted alkylene, a substituted alkenyl, a substituted alkynyl chain, a substituted or an unsubstituted alkyne chain, a substituted or an unsubstituted alkoxyl chain, an ether, an amine, an amide, a sulfonamide, an alkylamine, a thioether, a carboxylate, a polyethylene, a polypropylene, and a derivative or a combination thereof; and
X is (X)ₙ wherein n is an integer of from 1 to 50.

32. The antibody of claim 31 that is specific for programed cell death protein 1 (anti-PD1).

33. An antibody conjugated to one or more moieties of Y: 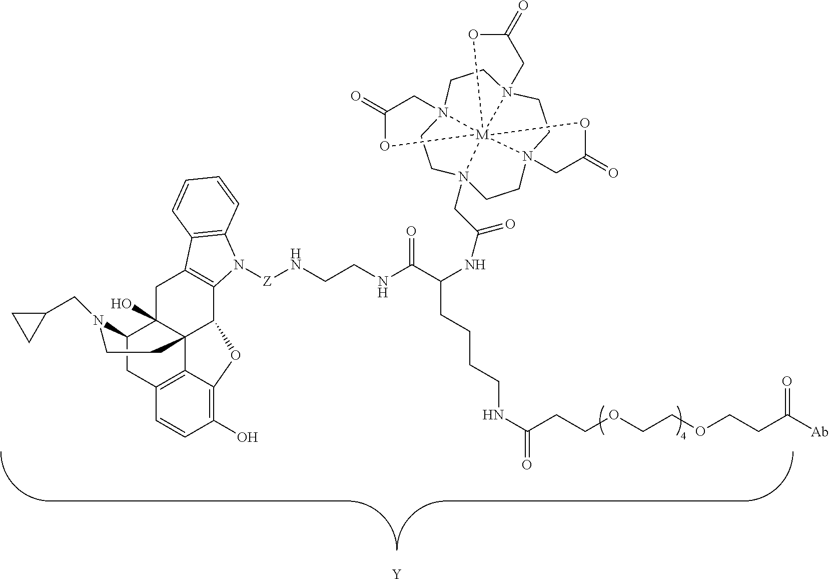
wherein Y is a delta opioid receptor targeting ligand;
Z is a linker fragment;
M is any metal of the lanthanide series of the Periodic Table;
wherein said linker fragment Z is a single atom or is selected form the group consisting a substituted carbon, an oxygen, a substituted or an unsubstituted sulphur, a substituted nitrogen, a substituted phosphorous, a substituted or an unsubstituted alkyl, a substituted alkenyl, a substituted alkynyl chain, a substituted or an unsubstituted alkylene, a substituted or an unsubstituted alkyne chain, a substituted or an unsubstituted alkoxyl chain, an ether, an amine, an amide, a sulfonamide, an alkylamine, a thioether, a carboxylate, a polyethylene, a polypropylene, and a derivative or a combination thereof;
Y is (Y)ₙ wherein n is an integer of from 1 to 50; and
Ab is an antibody.

34. The antibody of claim 33 that is specific for programed cell death protein 1 (anti-PD1).

35. The antibody of claim 31 wherein the antibody is specific for a programed cell death protein 1 (PD1), a PD-Li antagonist, or a CD137, an 0X40, or a CD40 agonist antibody.

36. The antibody of claim 33 wherein the antibody is specific for programed cell death protein 1 (PD1), a PD-L1 antagonist, or a CD137, an OX40, or a CD40 agonist antibody.

37. A method of treating cancer in a patient comprising administering to a patient a therapeutically effective amount of a composition of claim 1.

38. The method of claim 37 wherein said cancer is colon cancer.

39. The method of claim 37 wherein said cancer is lung cancer.

40. The method of claim 18, wherein the cancer is liver cancer.

41. The method of claim 39 wherein said lung cancer is small cell lung cancer and non-small cell lung cancer.

42. A method of decreasing peritoneal metastasis formation in a patient comprising: administering to a patient a therapeutically effective amount of a compound of claim 1.

43. The method of claim 42 wherein said metastasis is distal metastasis.
